(19) United States
(12) Patent Application Publication
(10) Pub. No.: US 2022/0064726 A1
(43) Pub. Date: Mar. 3, 2022

(54) METHOD FOR DETECTING POLYMERASE INCORPORATION OF NUCLEOTIDES

(71) Applicant: ILLUMINA, INC., San Diego, CA (US)

(72) Inventors: Ludovic VINCENT, San Diego, CA (US); Anmiv PRABHU, San Diego, CA (US)

(73) Assignee: ILLUMINA, INC., San Diego, CA (US)

(21) Appl. No.: 17/418,298

(22) PCT Filed: Aug. 18, 2020
(86) PCT No.: PCT/US2020/046854

§ 371 Date: Jun. 25, 2021

Related U.S. Application Data
(60) Provisional application No. 62/890,064, filed on Aug. 21, 2019.

(51) Int. Cl. C12Q 1/6874

(52) U.S. Cl. CPC C12Q 1/6874

(57) ABSTRACT

Provided is a method, including hybridizing test primers to immobilized primers, wherein the immobilized primers include a predetermined sequence of nucleotides and are attached to a substrate through their 5-prime ends, individual test primers are complementary to a portion of each of at least some of the immobilized primers, and no more than one test primer molecule hybridizes to an immobilized primer molecule, extending, by only one nucleotide, at least some of the test primers with a polymerase according to templates, wherein said templates comprise immobilized primers hybridized to said test primers and nucleotides incorporated into extended test primers comprise a fluorescent tag, and detecting an amount of fluorescent test primers.

### CROSS-REFERENCE TO RELATED APPLICATIONS

This application claims the benefit of U.S. Patent Application No. 62/890,064, filed Aug. 21, 2019 and entitled “Method for Detecting Polymerase Incorporation of Nucleotides,” the entire contents of which are incorporated by reference herein.

### SEQUENCE LISTING

The instant application contains a Sequence Listing which has been submitted electronically in ASCII format and is hereby incorporated by reference in its entirety. Said ASCII copy, created on Aug. 7, 2020, is named IP-1854-PCT_SL.txt and is 715 bytes in size.

### BACKGROUND

The majority of current sequencing platforms use “sequencing by synthesis” (SBS) technology and fluorescence based methods for detection. In a method, a substrate or surface includes one or more populations of primers attached, directly or indirectly, thereto, the primers having known sequences of nucleotides. A library or sample containing polynucleotides to be sequenced may be added thereto, with the polynucleotides of the library containing segments of stretches of nucleotides that are complementary to the primers attached to the substrate and thereby hybridizable thereto.

A polymerase enzyme may be added, with free single nucleotides, so as to extend the immobilized primers, being those attached to the surface or substrate, using the hybridized sample polynucleotides as a template. In a series of steps, copies of the template, sample polynucleotides are thereby added to the substrate as copies extending from the free ends of the primers immobilized on the surface or substrate. In subsequent steps, such copying and extending may be iteratively repeated to lead to the production of a plurality or a lawn of copies of the sample template polynucleotides, extending from what initially were immobilized primers. In a subsequent step, nascent strands may again be generated with a polymerase associated with such sample template nucleotides, now in the presence of fluorescently labeled nucleotides, under conditions where their addition to the nascent strand may be visualized so as to signal the identity of a given nucleotide being incorporated, permitting detection of the sequence of the nascent strand and, ultimately, sequencing of the sample polynucleotides.

In some examples, the amounts of a given sequence of primer or primers attached to the substrate may be determined before performing the foregoing hybridization, polymerization, and sequencing, to provide information as to the conditions present on a substrate or surface being used in the sequencing. Such information may be useful in interpreting results and assembling a sequence from the raw data generated. Also beneficial may be a method for detecting how well, or efficiently, a polymerase may be able to perform polymerization on primers near where they are attached to a substrate, accounting for steric hindrance or other effects that may influence enzyme activity. However, doing so according to a practice of extending the ends of the immobilized primers in a polymerization reaction with a fluorescent nucleotide and detecting addition of fluorescence to the immobilized primers modifies the primers, potentially altering subsequent hybridizing, polymerizing, and sequencing with the test sample polynucleotides whose sequence is sought. The present disclosure includes providing a method for measuring on-surface extension by a polymerase of one or more primers immobilized on a surface without requiring covalent modification of the immobilized primers, to permit subsequent use in SBS methodology.

### SUMMARY

In one aspect, provided is a method including hybridizing test primers to immobilized primers, wherein the immobilized primers include a predetermined sequence of nucleotides and are attached to a substrate through their 5-prime ends, individual test primers are complementary to a portion of at least some of the immobilized primers, and no more than one test primer molecule hybridizes to an immobilized primer molecule, extending, using one nucleotide, at least some of the test primers with a polymerase according to templates, wherein said templates include immobilized primers hybridized to said at least some of the test primers and nucleotides incorporated into said at least some of the test primers by the extending include one of a plurality of fluorescent tags, and detecting an amount of fluorescent test primers.

In an example, nucleotide sequences of a first plurality of the immobilized primers differ from nucleotide sequences of a second plurality of the immobilized primers. In another example, a first plurality of the test primers is complementary to a portion of the first plurality of immobilized primers, and a second plurality of the test primers is complementary to a portion of the second plurality of immobilized primers. In yet another example, first nucleotides incorporated into the first plurality of the test primers include a first of the plurality of fluorescent tags, second nucleotides incorporated into the second plurality of the test primers include a second of the plurality of fluorescent tags, and fluorescence emitted by the first of the plurality of fluorescent tags differs from fluorescence emitted by the second of the plurality of fluorescent tags.

In still another example, the substrate includes a metal oxide. In a further example, the substrate includes a metallic oxide and the metal oxide is selected from the group consisting of silicon dioxide, fused silica, tantalum pentoxide, titanium dioxide, aluminum oxide, hafnium oxide, and graphene oxide. In yet a further example, the substrate further includes a polymer. In still a further example, at least some of the immobilized primers are attached to the polymer. In an example, the polymer is a heteropolymer selected from

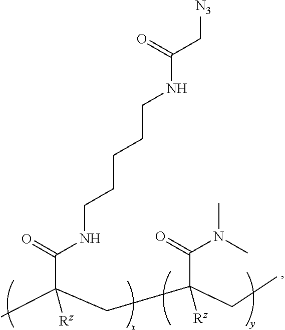

wherein x and y are integers representing a number of monomers and a ratio of x:y may be from approximately 15:85 to approximately 1:99, e.g., from approximately 10:90 to approximately 1:99, e.g., from approximately 10:90 to approximately 5:99, and

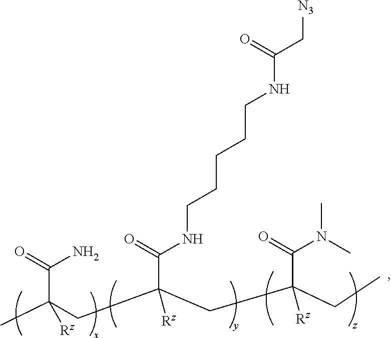

wherein x, y, and z are integers representing a number of monomers and a ratio of (x:y):z may be from approximately (85):15 to approximately (95):5, and wherein each R^{z} is independently H or C₁₋₄ alkyl. In some examples, a ratio of x:y:z may be from approximately 0:15:85 to approximately 0:5:95. In an example, a ratio of x:y is 5:95. In another example, a ratio of x:y:z is 5:85:10.

In another example, the polymerase is selected from a Klenow fragment and a Phi29 polymerase. In yet another example, the polymerase is attached to the substrate. In still another example, the polymerase is not attached to the substrate. In an example, the polymerase is selected from a Klenow fragment and a Phi29 polymerase. In a further example, the polymerase is attached to the polymer. In still a further example, the polymerase is not attached to the polymer.

In another example, detecting includes measuring fluorescence emitted from the test primers in response to a stimulus. In yet another example, test primers are hybridized to immobilized primers during the measuring. A further example includes dehybridizing the test primers from the immobilized primers before the measuring.

In an example, detecting includes measuring fluorescence emitted from test primers. For example, test primers may be hybridized to immobilized primers during the measuring. A further example includes dehybridizing the test primers from the immobilized primers before the measuring. A still further example includes comparing an amount detected of the first of the plurality of fluorescent tags to an amount detected of the second of the plurality of fluorescent tags.

In an example, a 5-prime end of at least some of the test primers does not overhang 3-prime ends of the immobilized primers. In another example, a 5-prime end of at least some of the test primers includes a 5-prime fluorescent tag having a 5-prime fluorescent tag spectrum, the 5-prime fluorescent spectrum differs from the fluorescent spectrum of the fluorescent tag of the nucleotide incorporated into the test primer by the extending, and detecting includes detecting an amount of 5-prime fluorescent tag and an amount of nucleotide fluorescent tag.

In a still another example, at least some of the test primers include overhanging test primers complementary to overhung immobilized primers, wherein 5-prime ends of the overhanging test primers overhang 3-prime ends of the overhung immobilized primers when hybridized thereto before the extending, further comprising extending, using one nucleotide, overhung immobilized primers, wherein nucleotides incorporated into overhung immobilized primers by the extending include an overhung fluorescent tag having an emission spectrum detectably different from an emission spectrum of the one of a plurality of fluorescent tags incorporated into the overhanging test primer by the extending, detecting an amount of fluorescent immobilized primers, and comparing an amount of fluorescent test primers to an amount of fluorescent immobilized primers.

In yet another example, at least some of the test primers include overhanging test primers complementary to overhung immobilized primers, wherein 5-prime ends of the overhanging test primers overhang 3-prime ends of the overhung immobilized primers when hybridized thereto before the extending and include a test primer 5-prime fluorescent tag, further comprising extending, using one nucleotide, overhung immobilized primers, wherein nucleotides incorporated into overhung immobilized primers by the extending include an overhung fluorescent tag, the test primer 5-prime fluorescent tag and the overhung fluorescent tag include a fluorescent tag pair when the overhanging test primers are hybridized to overhung immobilized primers after the extending of the overhung immobilized primers, and a combined fluorescence emitted by the fluorescent tag pair differs from a fluorescence emitted from the test primer 5-prime fluorescent tag and from a fluorescence emitted from the overhung fluorescent tag. In still a further example, detecting an amount of fluorescent test primers includes detecting the combined fluorescence emitted by the fluorescent tag pair.

It should be appreciated that all combinations of the foregoing concepts and additional concepts discussed in greater detail below (provided such concepts are not mutually inconsistent) are contemplated as being part of the inventive subject matter disclosed herein and contribute to the advantages and benefits as described herein.

### BRIEF DESCRIPTION OF THE DRAWINGS

These and other features, aspects, and advantages of the present disclosure will become better understood when the following detailed description is read with reference to the accompanying drawings, wherein:

FIG. 1 shows a flow diagram of an example of a method in accordance with aspects of the present disclosure.

FIG. 2 shows a flow diagram of an example of a method in accordance with aspects of the present disclosure.

FIG. 3 is a graphical representations of amounts of each of two different primers (P5 and P7) attached to a surface as assessed by quantifying amount of fluorescently tagged test primers hybridized thereto (y-axis), as a function of ratio of P5 primer to P7 primer included in reaction grafting primers to surface (x-axis), for each of three total concentrations of primer used in a reaction grafting primers to surface (left, middle, and right panels).

FIG. 4 is a graphical representations of measurement of amounts of two different primers (P5 and P7) attached to a surface as assessed by quantifying a ratio of an amount of fluorescently tagged test primers hybridized to P5 primers compared to P7 primers (y-axis), as a function of ratio of P5 primer to P7 primer included in reaction grafting primers to surface (x-axis), for each of three total concentrations of primer used in a reaction grafting primers to surface.

FIG. 5 is a graphical representations of amounts of each of two different polynucleotides complementary to primers (P5 and P7) attached to a surface, which polynucleotides were extended by a polymerase as assessed by quantifying amount of fluorescence incorporated into such test primers in accordance with aspects of the present disclosure (y-axis), as a function of ratio of P5 primer to P7 primer included in reaction grafting primers to surface (x-axis), for each of three total concentrations of primer used in a reaction grafting primers to surface (left, middle, and right panels).

FIG. 6 is a graphical representation of measurements of amounts of two polynucleotides complementary to primers (P5 and P7) attached to a surface, as assessed by quantifying a ratio of an amount of fluorescently tagged test primers hybridized to P5 primers compared to P7 primers (y-axis), as a function of ratio of P5 primer to P7 primer included in reaction grafting primers to surface (x-axis), for each of three total concentrations of primer used in a reaction grafting primers to surface.

FIG. 7 depicts percentages of hybridizable immobilized polynucleotides with associated polymerase activity in accordance with aspects of the present disclosure.

FIGS. 8A, 8B, and 8C depict examples of methods in accordance with the present disclosure wherein fluorescent nucleotides are added by a polymerase to polynucleotides complementary to primers attached to a surface.

### DETAILED DESCRIPTION

Provided is a method for assessing availability for polynucleotide primers attached to or immobilized on a surface or substrate for polymerase activity. Current SBS and related technologies employ numerous such primers to serve as initial points of polymerase reactions for determining nucleotide sequences of polynucleotides in a sample applied to said surface. Part of such methods include hybridization of sample oligonucleotides to surface-immobilized primers. Advantageously, an understanding of the total amount of primers available for hybridization to sample oligonucleotides may be determined. Such information may be useful for determining parameters to employ in use of such a service in an SBS or related method with such a surface. Such a method may further be useful in assessing whether different features of a surface with immobilized primers, or given polymerase enzymes, or different primer sequences attached to a surface, or any combination of the foregoing, affect polymerization at such primers, such as for use in an SBS or related method.

An SBS method may further include different species of primers hybridized to a surface, for hybridization to different portions of sample polynucleotides applied thereto. In an example, polynucleotides obtained from a sample, such as a tissue sample or other source of polynucleotides, may have been modified at their ends to include known sequences for hybridization to known primer sequences. For example, such polynucleotides may have a nucleotide sequence appended to one end that can hybridize to a first primer sequence and a nucleotide sequence appended to the other end that may hybridize to a second primer sequence. Furthermore, a surface for use in an SBS or related method may have affixed thereto two populations of primers, including the first primer sequence and the second primer sequence, whose sequences are hybridizable to one or the other end of the sample polynucleotides bearing the aforementioned modifications. Such sample polynucleotides may therefore hybridize to one or the other immobilized primer, or in some instances both. In some examples, a surface may have more than two populations of primers affixed thereto, and a sample of polynucleotides may have more than two nucleotide sequences complementary thereto affixed to each of one or another end, such as for hybridization to such surface-affixed primers. In other examples, individual polynucleotides in a sample may have appended sequences on each end that are the same as each other and therefore each hybridize to the same species of primer affixed to a surface, rather than bearing sequences at each and that differ from each other. In other examples, a sample may include polynucleotides some of which have end sequences appended that differ from each other, other polynucleotides with end-appended sequences that are the same as each other, polynucleotides with end-appended sequences that are different from end-appended sequences or some other polynucleotides in the sample, or different combinations of the foregoing.

For performing an SBS or related method on a surface to which, for example, populations of two or more such primers have been affixed, relative amounts of each population relative to the other affixed to the surface may be advantageously determined. For example, it may be desirable to create, or use, a surface to which equal proportions of a first and second primer have been affixed. For example, it may be desirable that sample polynucleotides modified so as to include a first and second sequence at a first and second end be equally likely to hybridize to a surface-affixed primer by one end or the other. Or, it may be desirable for polynucleotides in such a sample to hybridize to a greater degree to one primer than the other on the surface, or vice versa. Furthermore, polynucleotide molecules in a sample may bear unequal portions of end-appended nucleotide sequences, influencing likelihood of given polynucleotide molecules to hybridize to one or another surface-immobilized primer. Where polynucleotide hybridization to a surface-immobilized primer is employed for an SBS or similar or other method for polymerase-catalyzed elongation of a nucleotide strand, an ability to control and/or determine total and relative amounts of different primers affixed to the surface may be beneficial.

In some instances, surprisingly as disclosed herein, a total amount of primers, or of one or more species of primer, or relative amounts or ratios of primers, affixed to a surface, though useful information, may not be a perfect predictor for activity of polymerase activity relative to such primers immobilized on a surface. In some case, for example, it may be that a given primer is capable of serving as a more efficient initiation point for a polymerase reaction relative to another primer sequence hybridized to the same surface. In another example, introducing different variations to a surface to which primers are affixed may cause a disproportionate increase or decrease of polymerase activity at a given primer relative to another primer. In other examples, one polymerase molecule may be more efficient or more processive when initiating polymerization from a given surface-immobilized primer relative to a surface-immobilized primer of a different sequence. In other examples, distance from a surface or modified surface from which a primer affixed thereto extends, or distance of a portion of which primer a polymerase interacts with to initiate a polymerization reaction, may influence likelihood of polymerization being initiated, and perhaps disproportionately for one primer sequence as opposed to another. For all of the foregoing examples, and others, effects may be primer sequence-specific, meaning altering one variable or another may affect how or whether or how efficiently a polymerase initiates a polymerization reaction in relation to a surface-affixed primer of one sequence may differ from comparable affects for a surface-affixed primer of a different sequence.

In such cases, it is advantageous to determine not simply how many or relative proportions of one primer to another are affixed to a surface. In some examples, a given ratio of one primer to another may not have a one-to-one correspondence to how much polymerase activity may be initiated at a given primer. Where polymerization initiation probability, efficiency, processivity, or other parameters differ on a primer sequence-to-primer sequence basis, or other differences between species of primer on a surface such as perhaps distance a primer extends from the surface or distance the portion to which a polymerase binds to initiate polymerization extends from the surface, merely identifying relative amounts of primers affixed to a surface may not accurately reflect relative polymerization to initiate at each species of primer.

Polymerization at a primer affixed to a surface may be determined by performing a test polymerization reaction wherein one or more nucleotides is attached to the distal end of the primer, oriented away from the end proximal and attached to the surface, by a polymerase. However, such a method involves covalently modifying the surface-affixed primer, namely by covalently attaching a nucleotide to the free end. In some examples, as provided in the present disclosure, it may be desirable to measure polymerization initiated at a surface-immobilized primer without including the making of covalent modifications to the surface-affixed primer. For example, it may be desirable to test or confirm levels or relative levels of polymerase activity of a species of primer or species of primers affixed to a surface before using such surface in an SBS method or similar method. Or, serial assays may be desirable to run on a given surface with primers affixed thereto and direct comparison of outputs of such serial assays to each other may be desirable, without being confounded by covalent modifications having been made to primers by one or more of the serial assays.

Polynucleotides have so-called 3-prime and 5-prime ends, referring to the numbered carbon on the sugar ring of the nucleotide at each end of the strand, not directly attached to an adjoining nucleotide in the sequence via such carbon. Polymerases add nucleotides to a growing strand in the 5-prime to 3-prime direction. That is, the 5-prime carbon of the sugar of a free nucleotide is attached, through an intervening phosphate group, to the 3-prime sugar of the nucleotide on the so-called 3-prime end of the polynucleotide, by a reaction catalyzed by a polymerase enzyme. In some examples of surfaces to which primers have been affixed for use in performing SBS or other related methods, primers may be attached to or immobilized on a substrate oriented with their 5-prime ends towards and proximal to the surface or substrate, with the 3-prime ends distal and free. In such examples, a sample polynucleotide may hybridize to such primer. In the presence of a polynucleotide and free nucleotides, the primer may therefore be extended, starting with the addition of nucleotides to the free, 3-prime end, using hybridized sample polynucleotide as a template. Polymerization may continue with extension of a nascent nucleotide extending from the initial free 3-prime end of the surface-affixed primer. Conventional SBS and related technologies may employ such method as part of a process of ultimately determining a sample polynucleotides sequence.

As explained above, in some cases it may be advantageous to quantify how much of a given primer species is present affixed to a surface, or relative amounts of different species of primer. Conventionally, one way of doing so is to perform a first test where polynucleotides are added to a surface, bearing surface-affixed primers, wherein the polynucleotides are complementary to at least a portion, or sequence within, the or some of the primers. Such polynucleotides may include a marker to permit detection thereof. Conventionally, for example, short polynucleotides of a length of, for example, 10 to 60 nucleotides, complementary to a portion, or sequence within, primers affixed to a surface may be added to the surface so as to hybridize to primers. The polynucleotides may be modified such as by including a fluorescent probe or molecule. Where more than one species of primer, such as two species of primer, are affixed to a surface, two different polynucleotides, one hybridizable to one species of primer and the other hybridizable to the other species of primer, may be added to the surface and incubated therewith.

Each species of polynucleotide may include a fluorophore and each fluorophore may fluoresce in a manner that enables differentiation of one fluorophore from the other, such as by having different emission spectra from each other. Following incubation of a surface with such polynucleotides to permit their hybridization to respective, complementary primers, unhybridized polynucleotides may be washed away and fluorophore remaining on the surface, reflecting an amount of one or the other or both species of polynucleotide present and, thus, complementary primer affixed to the surface. Hybridized fluorescent polynucleotides may then be dehybridized from the surface-affixed primers, resulting in a surface without covalent modification from its state prior to hybridization with fluorescent polynucleotides, while information as to the amount and/or relative amount of primers and/or different species of primers hybridized thereto having been obtained for future reference.

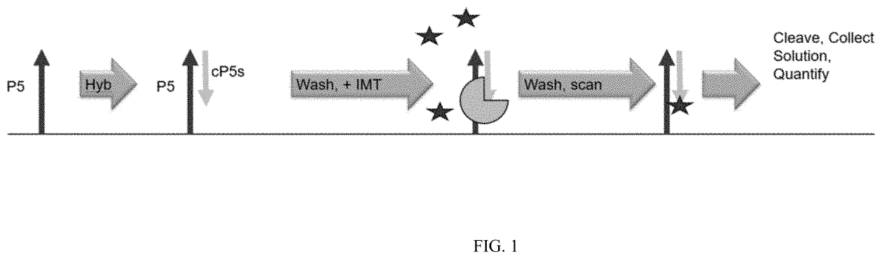

A different or additional method, in accordance with aspects disclosed herein, may be employed for determining polymerase activity potential on a primer and/or species of primers affixed to a surface, in some examples without covalently modifying the surface-affixed primers. An example is depicted in FIG. 1. In this example, immobilized primers are affixed to a surface. In this example, an immobilized primer is identified as P5, with its 3-prime end indicated by an arrowhead, distal to the surface, and its 5-prime end proximal to and oriented towards the surface or substrate to which the immobilized primer is affixed or immobilized. The immobilized primer's sequence is known or predetermined, or at least a portion of it is known or predetermined, such that a polynucleotide may be designed to have a sequence of nucleotides complementary thereto such that the polynucleotide may hybridize to the primer or at least a portion of the primer. Furthermore, the primer's sequence of nucleotides or portion therefore may be known such that the identity nucleotides to which a polynucleotide may not directly hybridize, but adjacent by one, or more nucleotides to immobilized primer nucleotides to which a complementary nucleotide may hybridize, are known.

Referring again to FIG. 1, a hybridization is performed, including adding test primers to the surface, such as in a hybridization solution, in which such test primers may hybridize to portions of immobilized primers to which they are complementary. In the example illustrated in FIG. 1, a polynucleotide complementary to the P5 immobilized primer, indicated by cP5, is shown hybridized to a portion of the P5 immobilized primer. In this example, the arrowhead on the cP5 polynucleotide indicates the 3-prime end of the cP5 test primer. Whereas the 5-prime end of the P5 immobilized primer is proximal to the surface, in accordance with base pairing complementarity, the 3-prime end of the complementary cP5 polynucleotide is proximal to the surface and its 5-prime end distal thereto when the complementary cP5 primer is hybridized to the immobilized P5 primer. Furthermore, in this example, the 5-prime end of the complementary cP5 primer does not overhang the 3-prime end of the P5 immobilized primer. That is, the 3-prime terminal of the P5 immobilized primer is complementary and hybridized to a nucleotide of the complementary cP5 polynucleotide.

In this configuration, a polymerase may add a nucleotide to the complementary cP5 polynucleotide but not to the P5 immobilized primer. That is, a polymerase uses an unhybridized nucleotide, 5-prime to the 5-prime-most nucleotide of a template polynucleotide hybridized to a polynucleotide to be extended by the polymerase, as a template for addition of the next nucleotide to be added by the extending. In the example shown in FIG. 1, there is no such 5-prime nucleotide of the complementary cP5 polynucleotide. That is, the 5-prime most nucleotide of the complementary cP5 polynucleotide is hybridized to a nucleotide of the P5 immobilized primer. Thus, there is no nucleotide of the complementary cP5 polynucleotide that may be used as a template for addition of another nucleotide to the 3-prime end of the immobilized primer by a polymerase. The complementary cP5 polynucleotide may be extended by a polymerase, however. 5-prime adjacent to the nucleotide of the P5 immobilized primer that is hybridized to the 3-prime most nucleotide of the complementary cP5 primer is a nucleotide of the P5 immobilized primer that is not hybridized to a nucleotide of the complementary cP5 polynucleotide. A polymerase brought into contact with the hybridized P5 immobilized primer and the complementary cP5 polynucleotide may extend the 3-prime end of the complementary cP5 nucleotide by at least one nucleotide, the one nucleotide being complementary to the nucleotide of the P5 immobilized primer that is one nucleotide 5-prime to the nucleotide of the P5 primer that is hybridized to the 3-prime end of the complementary cP5 polynucleotide. In this example, only one complementary cP5 polynucleotide hybridizes to a given P5 immobilized primer.

Continuing with FIG. 1, after hybridization of complementary cP5 nucleotides to P5 immobilized primers, unhybridized complementary cP5 polynucleotides may be washed away. The surface may next be contacted with polymerase enzymes, such as in a polymerization solution. Also included in the solution may be nucleotides that may be added to the 3-prime end of the complementary cP5 polynucleotide by a polymerase, in accordance with the foregoing. In an example, the only polynucleotides included in the polymerization solution may be complementary to the 5-prime next, non-hybridized nucleotide of the P5 immobilized primer, such that a polymerase may catalyze their addition to the 3-prime end of the complementary cP5 polynucleotide, using the P5 immobilized primer as a template. In some examples, the following 5-prime nucleotide of the P5 immobilized primer is known to be different from the P5 nucleotide that served as a template for addition of a nucleotide to the 3-prime end of the complementary cP5 polynucleotide. In such examples, when only one species of nucleotide is included in the polymerization solution, being attachable to the 3-prime end of the complementary to the cP5 polynucleotide by a polymerase using the P5 immobilized primer as a template, only one nucleotide is accordingly attached to the complementary cP5 polynucleotide.

In the example illustrated in FIG. 1, a hybridization polymerization process shown as being performed separately from each other. However, in other examples, test polynucleotides complementary to immobilized primers, polymerase enzyme, and nucleotides for incorporation by a polymerase, may all be added to the surface in a single solution for hybridization and polymerization to occur in the same solution.

In other examples, nucleotides in the polymerization reaction may be modified such that only one may be added to a nascent strand in a polymerization reaction, absent further intervention. For example, modifications at the 3-prime end of a nucleotide or related molecule may prevent an ability for another nucleotide to be added thereto once the nucleotide has been added to the 3-prime end of a growing strand by a polymerase. For example, a nucleotide may have a chemical modification at its 3-prime carbon such as addition of an azidomethyl or other group that may prevent further extension of the strand after said nucleotide is added. In other examples, nucleotides in the polymerization solution may be dideoxynucleotides, lacking hydroxyl groups on the 3-carbon, thus lacking an attachment site for the 5-prime phosphate-carbon of a next nucleotide. In other examples, only two, only three, only four, only five, only six, only seven, only eight, only nine, only ten, only eleven, only twelve, only thirteen, only fourteen, only fifteen, only between fifteen and twenty, or more nucleotides may be added to a 3-prime end of a complementary cP5 polynucleotide. An amount of such added nucleotides may be controlled by controlling which species of nucleotide is included in the polymerization reaction, such as by including all nucleotides complementary to available template P5 immobilized primer nucleotides except to the P5 immobilized primer that is one nucleotide 5-prime to the last P5 immobilized primer intended to serve as a template for extension of the 3-prime end of the cP5 polynucleotide. Or the last nucleotide for addition to the extended 3-prime end of the complementary cP5 polynucleotide may be modified to prevent further extension therefrom according to the above or other examples.

In another example, more than one species of immobilized primer may be attached to the surface or substrate, having different sequences from each other. Furthermore, different species of test primers may be incubated with the surface or substrate so as to permit hybridization of the species of test primers complementary to one species of immobilized primer to that species of immobilized primer and polynucleotides complementary to a second species of text primer to hybridize to said second species of immobilized primer. In an example, a or each species of test primer is hybridizable only to one species of immobilized primer and the other or another species of test primer is complementary only to another species of immobilized primer, such that only one species of test primer will hybridize to a given species of immobilized primer. In another example, species of test primers may by hybridizable to both or all species of immobilized primers affixed to a substrate or surface.

In another example, whether or not one or more species of test primer incubated with a surface is hybridizable to only one or more than one species of surface-immobilized primer, test primer may be designed so that only one species of nucleotide may be added by a polymerase to the 3-prime end of a test primer hybridized to one species of test primer and only another species of nucleotide may be added by a polymerase to the 3-prime end of another test primer hybridized to another species of immobilized primer. For example, in either or both cases, the 5-prime next nucleotide of each species of immobilized primer immediately 5-prime to the 5-prime most immobilized primer nucleotide hybridized to the 3-prime end of a test primer to which it is hybridized may be different from the comparable nucleotide of the other species, or of another species if there are more than two species total, of immobilized primer. In such cases, a polymerase may catalyze the addition of one species of complementary nucleotide to the 3-prime end of one test primer hybridized to one species of immobilized primer and catalyze the addition of a different species of complementary nucleotide to the 3-prime end of another test primer hybridized to another species of immobilized primer.

Thus, a different nucleotide may be added to test primers hybridized to different immobilized primers. In an example, different species of nucleotides are identifiable in a manner that permits differentiation between different nucleotides. For example, different species of nucleotides may possess different markers to which they are covalently attached. In an example, nucleotides may possess a fluorescent marker, observable by fluorescent imaging. Two species of nucleotides may possess two different species of fluorophore, each possessing a different emission spectrum from the other, such as being excitable by a different wavelength of light or other electromagnetic radiation compared to the other and/or emitting a detectably different wavelength from the other upon excitation. Numerous fluorophores are used in the relevant field for differentiating between different nucleotides, including in various examples of SBS and related methods. According to this example, with different species of nucleotides being added to test primers complementary to different species of immobilized primers affixed to a surface, and different fluorophores, for example, attached to different nucleotides, polymerase activity in connection with different species of immobilized primers may be determined. One species of fluorescent or other marker may be detected on the surface where test primers are hybridized to immobilized primers.

With test primers and immobilized primers designed such that the species of nucleotides that may be added to a given test primer when hybridized to an immobilized primer in the presence of nucleotides and a polymerase in a polymerization solution, and the detection characteristics of markers such as fluorophores affixed to different species of nucleotides also known, detection of a given marker may indicate the species of primer at which a polymerization reaction is catalyzed by a polymerase. Where more than one species of immobilized primer and/or more than one species of test primer is used in accordance with the disclosed method for assessing polymerase activity at primers immobilized on a substrate, two different species of test primers may be simultaneously incubated with and hybridized to immobilized primers immobilized on a surface and the species of test primers simultaneously subjected to extension during the same polymerization reaction as each other. In other examples, one species of test primer may be incubated at a time, and/or a species of nucleotide that may be added to a test primer when hybridized to only one of multiple immobilized primers may be present in a given polymerase reaction. Subsequently, a second incubation process with the or another species of test primer may be conducted, and/or another polymerization reaction with a species of nucleotide attachable to a polynucleotide when hybridized to a second species of immobilized primer, may be conducted.

In an example, species of nucleotide incorporated by one or more polymerization reaction may be detected while test primers that have been extended by the addition of a detectable nucleotide as disclosed herein may be detected while the test primers remain hybridized to the immobilized primers. For example, referring to FIG. 1, a polymerization reaction is indicated by incubation of test primer hybridized to immobilized primer with nucleotides (shown as star shapes) and a polymerase (represented as a three-quarter circle shape. In this example, a polymerase is shown as being in solution. But, in another example, a polymerase may be bound to a substrate. After a polymerization reaction, unbound polymerase, unbound test primer, and/or free nucleotide may be washed away under hybridization conditions that permit continued hybridization of test primers to immobilized primers. The surface may then be scanned, according to known methods, for detection of the detectable nucleotides on the surface. Locations and/or overall amounts of each species of nucleotide present after washing unincorporated nucleotide and unhybridized test primer from the surface indicates polymerization that occurred at a given immobilized primer. In some examples, detection, measurement, or quantification of incorporated nucleotide may occur after test primers that were extended during a polymerization reaction have been dehybridized from immobilized primers, such as following incubation in a rehybridization solution. In such examples, rather than, or after, measuring incorporated nucleotide amounts and/or location when extended test primers remained hybridized to immobilized primers, an amount of incorporated nucleotide may be measured in, for example, a dehybridization solution containing extended test primers that were dehybridized from immobilized primers following polymerase-catalyzed nucleotide incorporation.

The example illustrated in FIG. 1 is but one, non-limiting example. Many modifications to or variations of the example illustrated in FIG. 1, including such as were described in the foregoing paragraphs, are also included in the present disclosure. Moreover, only one species of immobilized primer and only one species of test primer and only one species of detectable nucleotide are depicted in FIG. 1. In keeping with the examples described above, an example may include multiple species of immobilized primer. An example may include multiple species of test primer. An example may include multiple species of labelled, detectable nucleotide for incorporation into test primers by a polymerase. As may further be understood, many copies of one or more species of immobilized test primer may be attached to a surface, on the order of thousands or hundreds of thousands or millions or tens of millions, or more, including on the order of up to 1×10¹¹ primers per mm² of surface are of the surface. Only one molecule of immobilized primer is illustrated in FIG. 1 for purposes of illustration.

In an example, one species of test primer, hybridizable to one species of immobilized primer, may correspond to a sequence at or added to ends of nucleotides in a sample whose sequence is to be determined in an SBS run using the surface on which the method disclosed herein is used. Another species of test primer, hybridizable to another species of immobilized primer, may correspond to a sequence at or added to the other ends of nucleotides in a sample whose sequence is to be determined in an SBS run using the surface on which the method disclosed herein is used. Thus, for example, immobilized primers such as a P5 primer and a P7 primer may be immobilized primers on a surface. Furthermore, test primers used in a method as disclosed herein may have sequences that are complementary to P5 and P7 immobilized primers, and sample polynucleotides whose sequence is to be interrogated during a subsequent SBS run using the surface or substrate may have sequences corresponding to such test primers added to one and/or the other end thereof as part of such subsequent SBS or similar processing.

Polymerase activity determined in accordance with a method as disclosed herein in an example when one or more test primer sequence used corresponds to sequence of polynucleotides added to ends of sample polynucleotides may provide predictive information about hybridization of such sample polynucleotides to such immobilized primers and/or polymerase activity to be predicted at such points of hybridization. More generally, irrespective of any subsequent SBS or related methodology performed on a substrate with which the method as disclosed herein has been used, information obtained with the method as disclosed herein indicates polymerase activity at surface and, potentially, differentiable polymerase activity depending on a given immobilized primer. Surprisingly, as disclosed herein, quantifying immobilized primers to which test primers are capable of hybridizing, without more, does not have a one-to-one correspondence to polymerization at different immobilized primer sites.

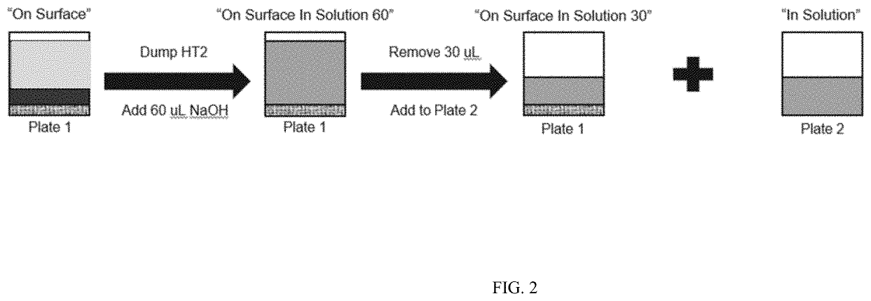

Non-limiting examples for methods for quantifying nucleotides incorporated by a polymerase reaction in accordance with the present disclosure are depicted in FIG. 2. As disclosed above in some examples amounts of incorporated nucleotide, detectable for example by a known fluorescence emission spectrum, may be detected while test primers into which they were incorporated during a polymerization reaction are still hybridized to immobilized primers attached to a surface, as indicated on plate 1 on the left side of FIG. 2. In another example, the marker affiliated with an incorporated nucleotide me be released from an immobilized primer. For example, test primers hybridized to immobilized primers and containing incorporated nucleotides with a detectable marker such as a fluorophore may be dehybridized from immobilized primers. Or, detectable markers such as fluorophores may be chemically cleaved from test primers and thereby be released into solution. In either such case, fluorescence or other marker indicia as relevant in solution may be detected, rather than (or after) detection on surface before release of marker from immobilized primer. Such an example is illustrated in the central plate 1 panel in FIG. 2.

In still another example, solution containing fluorophore or other markers after release from immobilized primers may be removed from the solution on the surface and transferred to another detection vessel for measurement in a different marker, such as fluorescent, detection system or apparatus. Such an example is shown in the right-hand panel of FIG. 2, where some of the solution from plate 1 has been removed and placed in, in this example, plate 2. However, the second vessel for measurement need not be a plate but may be any known apparatus or system or method for detection. In an example, gel electrophoresis may be used to separate and then visualize test primers to which marker-attached nucleotides have been added in a polymerase reaction as described. In other examples where gel electrophoresis is used to resolve test primers including nucleotides incorporated in a polymerase reaction as disclosed, differentiation between polymerase activity associated with one species of immobilized primer-test primer hybridized pair and another species of immobilized primer-test primer hybridized pair may be based on differences in lengths of respective test primers following the polymerase-catalyzed nucleotide incorporations. For example, one test primer species may be longer than the other, whereas the same amount of nucleotides may be added to each (such as one) or in any event an amount may be added to each such that the total length of each after polymerase-catalyzed nucleotide addition may be sufficiently different to permit separation by gel electrophoresis. This may be possible even if nucleotides incorporated into each species of test primer did not include markers that were detectably differentiable from one another. In some examples, where gel electrophoresis or other size-based resolution methods may be used, the incorporated nucleotides or one of them may lack independently detectable marker altogether.

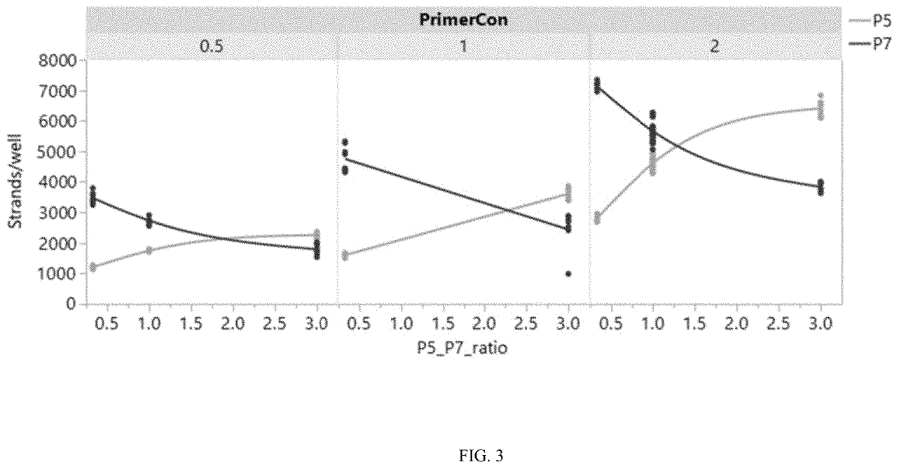

Surprisingly, and as disclosed herein, measuring test primer hybridization to immobilized primers may not indicate that or whether polymerase activity at different species of immobilized primer-test primer hybridization pairs is equivalent or if not how they differ from each other. Examples of possible differences in such polymerase activity may not necessarily be reflected in FIGS. 3-6. FIG. 3 shows amounts of test primers hybridized to immobilized primers on a surface (Y-axis), expressed as an amount of strands per well, where strands indicates an extrapolation of amount of immobilized primers from an amount of fluorescence detected and well indicates a portion of surface from which measurement was taken. Fluorescence is fluorescence detected from test primers including a fluorophore. In this example, two species of immobilized primer were used, P5 and P7 and, thus, two species of test primer complementary thereto used, cP5 and cP7. cP5 and cP7 included fluorescent markers that may be detected differentially one from the other. Three panels are shown. Each panel reflects a different total concentration of primer used in a reaction in which primers were attached to a surface or substrate to become immobilized primers, according to known methods. Total concentration from left panel to right panel is indicated in μM (0.5 μM, 1 μM, and 2 μ). On the x-axis is indicated the relative ratio of P5 primer to P7 primer included in the reaction in which primers were attached to a surface or substrate to become immobilized primers, according to known methods. Thus, FIG. 3 demonstrates, for different total concentrations of primer included in a reaction for affixing primers to a surface, and for different relative concentrations of each primer given such total primer concentrations, how many immobilized primers can be hybridized to with complementary test primers designed to hybridize thereto.

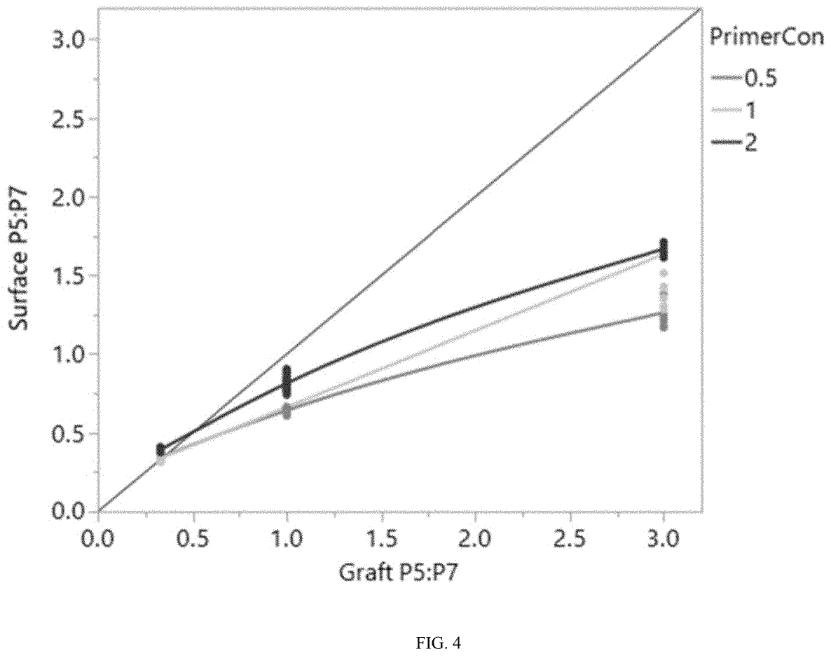

These data are expressed in a different format in FIG. 4. In FIG. 4, the x-axis, as the x-axis in FIG. 3, is the relative ratio of P5 primer to P7 primer included in the reaction in which primers were attached to a surface or substrate to become immobilized primers, according to known methods. The amount of fluorescence detected on the surface corresponding to each of P5 and P7 immobilized primers was determined (cf. the y-axis of FIG. 3), and a ratio of P5-related fluorescence to P7-related fluorescence was calculated for each relative ratio of P5 to P7 included in the immobilized primer immobilization reaction used to affix immobilized primers to the substrate. This ratio of P5- to P7-related fluorescence is given on the y-axis of FIG. 4. The three plot lines indicate the three different total concentrations of primer used in the reaction in which primers were attached to a surface or substrate to become immobilized primers, according to known methods (0.5 μM, 1 μM, and 2 μM, as in the three panels shown in FIG. 3). A 1:1 correspondence line is shown as a prediction of plots that may have been expected in equimolar concentrations of primers used in a reaction when immobilizing primers to a surface lead to equimolar amounts of fluorescent test primer hybridizing to the surface. However, as shown, actual values fall below the predicted 1:1 line. In other words, at these total concentrations, increasing a ratio of P5 to P7 in the immobilization reaction did not correspond to an equivalent increase in an amount of P5 available for hybridization by test primers relative to P7 immobilized primers (e.g., less than proportionate P5 to P7 grafting to the surface).

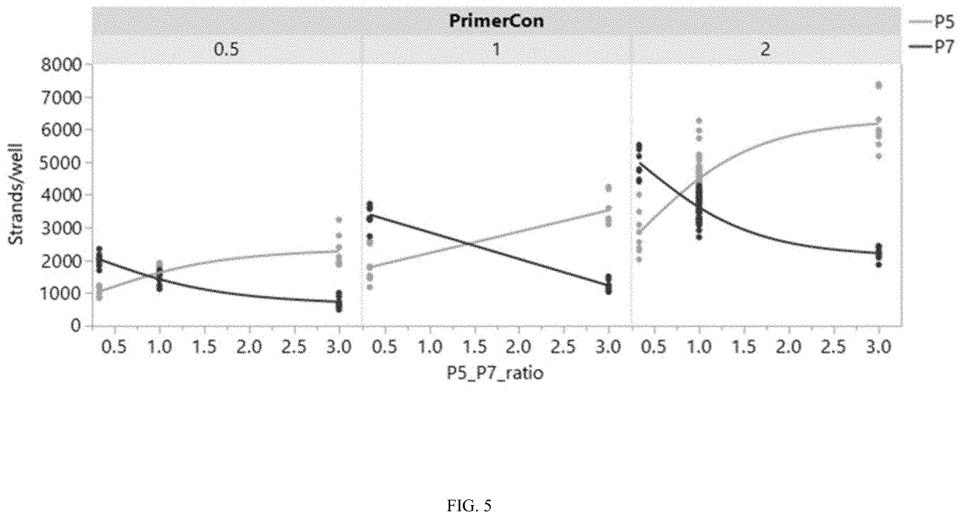

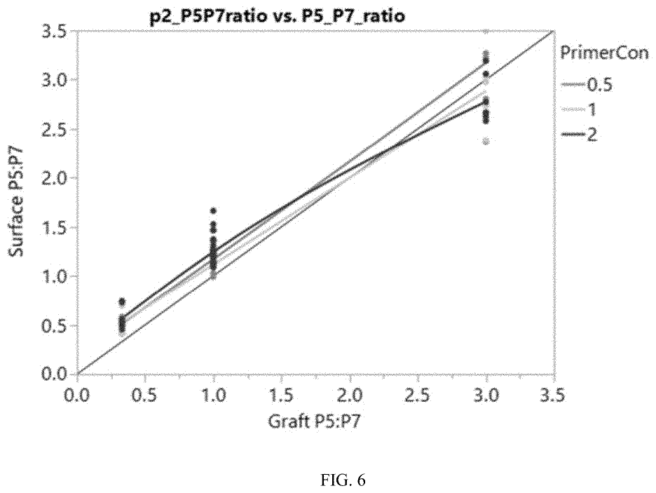

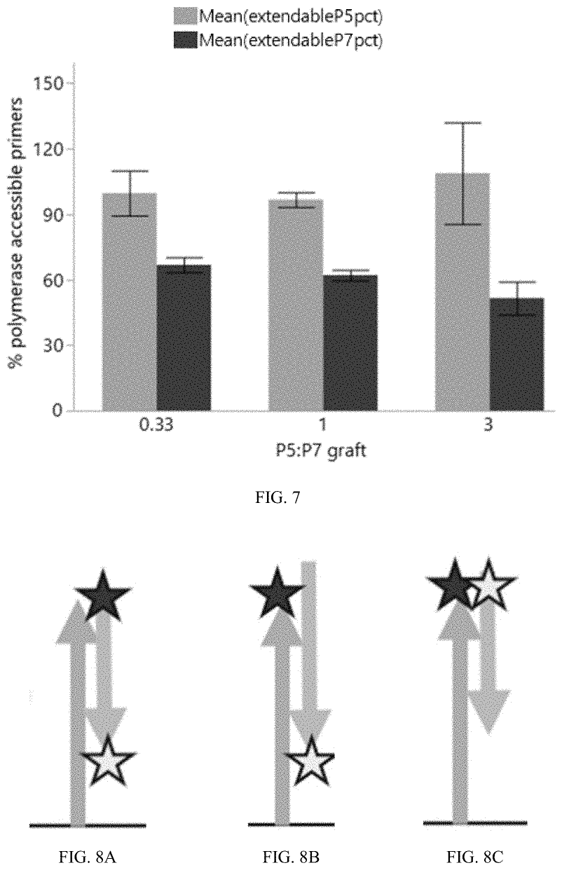

FIGS. 5 and 6 are comparable to FIGS. 3 and 4 except that the y-axes, rather than reporting an amount of fluorescent test primer hybridized to immobilized primers, shown are amounts of fluorescent nucleotide attached to the 3-prime end of a polynucleotides complementary to P5 or P7 in accordance with a method as disclosed herein (e.g., see FIG. 1). Other aspects of FIGS. 3 and 4 are applicable to FIGS. 5 and 6. The three panels in FIG. 5 correspond to the total concentrations of primer used when immobilizing primers to a surface, and the x-axis indicates relative concentrations of P5 to P7 in said reactions. In FIG. 6, the x-axis is as described for FIG. 4. The y-axis is similar as to that described for FIG. 4 except that the ratio is not of P5 to P7 hybridization but instead of P5 to P7-related polymerase activity. Again, a 1:1 plot is drawn indicating where results may fall if increasing a relative concentration of P5 to P7 in a reaction during which primers were immobilized to a surface yields a commensurate increase in polymerization associated with P5 immobilized primers relative to polymerization associated with P7 immobilized primers.

In this case, surprisingly, increasing P5 relative concentration did yield a commensurate increase in P5-related polymerase activity. Note, however, that, as indicated in FIG. 4, increasing relative concentration of P5 did not lead to a commensurate increase in P5 hybridization to a test primer. In other words, in combination, FIGS. 4 and 6 show that polymerase activity associated with P5 primers may increase above and more than what a measurement of P5 hybridization availability on its own may lead one to predict. Rather, unexpectedly and as surprisingly disclosed herein, increasing P5:P7 ratio proportionately caused an increase in P5:P7-related polymerase activity despite a less than proportionate increase in P5:P7 hybridization availability. These differences are illustrated in FIG. 6. FIG. 6 shows, for three different relative concentrations of P5 primer to P7 used in a reaction to immobilize primers to a surface, a percentage of polymerase accessible primer relative to a total amount of hybridization-accessible primer (e.g., grafted primer). As can be seen, in this example, a higher percentage of P5 primers are polymerase activity accessible relative to P7 primers.

Such information may be beneficial. It can be used to determine conditions to apply in a grafting reaction during which primers are immobilized to a surface so as to arrive at predetermined hybridization accessibility and polymerase activity accessibility. In other examples, comparative polymerase accessibility of different primers, of any desired sequence, may be assayed. Primers of different lengths may be assayed. Different concentrations of immobilized primers on a surface may be tested. Test primers may be assayed. Different polymerases may be assayed, as well as different substrate. In some examples, a surface of a substrate may possess different surface modifications. A method as disclosed herein may be used to assay different variations to a surface and effects that they may have on different immobilized primer polymerase activity availabilities.

Any of a variety of polymerases can be used in a method set forth herein including, for example, protein-based enzymes isolated from biological systems and functional variants thereof. Reference to a particular polymerase, such as those exemplified below, will be understood to include functional variants thereof unless indicated otherwise. A particularly useful function of a polymerase is to catalyze the polymerization of a nucleic acid strand using an existing nucleic acid as a template. Other functions that are useful are described elsewhere herein. Examples of useful polymerases include DNA polymerases and RNA polymerases, functional fragments thereof, and recombinant fusion peptides including them. Example DNA polymerases include those that have been classified by structural homology into families identified as A, B, C, D, X, Y, and RT. DNA Polymerases in Family A include, for example, T7 DNA polymerase, eukaryotic mitochondrial DNA Polymerase gamma., E. coli DNA Pol I (including Klenow fragment), Thermus aquaticus Pol I, and Bacillus stearothermophilus Pol I. DNA Polymerases in Family B include, for example, eukaryotic DNA polymerases a, 6, and E; DNA polymerase C; T4 DNA polymerase, Phi29 DNA polymerase, Thermococcus sp. 9°N-7 archaeon polymerase (also known as 9°N™) and variants thereof such as examples disclosed in U.S. Patent Application Publication No. 2016/0032377 A1, and RB69 bacteriophage DNA polymerase. Family C includes, for example, the E. coli DNA Polymerase III alpha subunit. Family D includes, for example, polymerases derived from the Euryarchaeota subdomain of Archaea. DNA Polymerases in Family X include, for example, eukaryotic polymerases Pol beta, Pol sigma, Pol lambda, and Pol mu, and S. cerevisiae Po14. DNA Polymerases in Family Y include, for example, Pol eta, Pol iota, Pol kappa, E. coli Pol IV (DINB) and E. coli Pol V (UmuD'2C). The RT (reverse transcriptase) family of DNA polymerases includes, for example, retrovirus reverse transcriptases and eukaryotic telomerases. Example RNA polymerases include, but are not limited to, viral RNA polymerases such as T7 RNA polymerase; Eukaryotic RNA polymerases such as RNA polymerase I, RNA polymerase II, RNA polymerase III, RNA polymerase IV, and RNA polymerase V; and Archaea RNA polymerase. Other polymerases, for example as disclosed in U.S. Pat. No. 8,460,910, are also included among polymerases as referred to herein, as are any other functional polymerases including those having sequences modified by comparison to any of the above mentioned polymerase enzymes, which are provided merely as a listing of non-limiting examples.

The term “surface” or “substrate” refers to a support or substrate upon which test primers may be attached. The surface may be a wafer, a panel, a rectangular sheet, a die, or any other suitable configuration. The surface may generally be rigid and insoluble in an aqueous liquid. Examples of suitable surfaces include epoxy siloxane, glass and modified or functionalized glass, polyhedral oligomeric silsequioxanes (POSS) and derivatives thereof, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon, ceramics/ceramic oxides, silica, fused silica, or silica-based materials, aluminum silicate, silicon and modified silicon (e.g., boron doped p+silicon), silicon nitride (Si3N4), silicon oxide (SiO2), tantalum pentoxide (TaO5) or other tantalum oxide(s) (TaOx), hafnium oxide (HaO2), aluminum oxide, graphene oxide, titanium dioxide, carbon, metals, inorganic glasses, or the like. The surface may also be glass or silicon or a silicon-based polymer such as a POSS material, optionally with a coating layer of tantalum oxide or another ceramic oxide at the surface. A surface or substrate may include or be silicon or one or more other transition metals.

Immobilized primer sequences and test primer sequences may be any suitable sequence in accordance with the above disclosure. In an example, sequence of a P5 immobilized primer as disclosed herein is represented by SEQ ID NO: 1 (CAAGCAGAAGACGGCATACGAGAT), sequence of a P7 immobilized primer as disclosed herein is represented by SEQ ID NO: 2 (AATGATACGGCGACCACCGAGATCTACAC). Test primers for hybridization may be sequences complementary to portions of these sequences.

Fluorescence may be detected by any suitable methodology. For example, fluorescence may be detected on surface using known optical fluorescence detection methodology, where fluorescence is elicited on and detected from surface where hybridized polynucleotide remains hybridized to template. In another example, hybridized test primer may be dehybridized from immobilized primer and eluted in solution, with fluorescence detected in said elution solution rather than on surface. Any of various known methods for measuring fluorophores commonly attached to nucleotides for use in the disclosed method may be employed, on surface, in solution, or otherwise.

Detection can be carried out by any suitable method, including fluorescence spectroscopy or by other optical means. The fluorescent label may be a fluorophore, which, after absorption of energy, emits radiation at a defined wavelength. Many suitable fluorescent labels are known. For example, Welch et al. (Chem. Eur: J. 5(3): 951-960, 1999) discloses dansyl-functionalised fluorescent moieties that can be used in the present invention. Zhu et al. (Cytometry 28:206-211, 1997) describes the use of the fluorescent labels Cy3 and Cy5, which can also be used according to aspects of the present disclosure. Labels suitable for use are also disclosed in Prober et al. (Science 238:336-341, 1987); Connell et al. (BioTechniques 5(4):342-384, 1987), Ansorge et al. (Nucl. Acids Res. 15(11):4593-4602, 1987) and Smith et al. (Nature 321:674, 1986). Other commercially available fluorescent labels include, but are not limited to, fluorescein, rhodamine (including TMR, Texas red and Rox), alexa, bodipy, acridine, coumarin, pyrene, benzanthracene and the cyanins. Any suitable modification of any of the foregoing may be adopted for use in and employed in accordance with the method as disclosed herein. A fluorescent nucleotide may include such an attachment, for example. Commercially available fluorescently tagged nucleotides may be used in accordance with the present disclosure. A non-limiting, generalized example of fluorescent nucleotide may be depicted as follows:

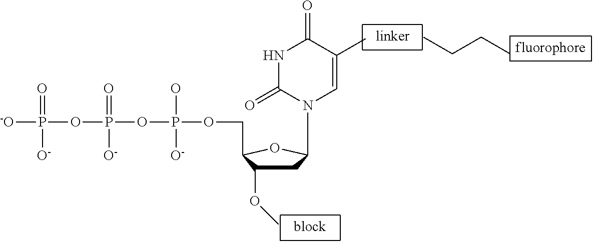

### EXAMPLES

As used herein, the term “nucleotide” is intended to include natural nucleotides, analogs thereof, ribonucleotides, deoxyribonucleotides, dideoxyribonucleotides and other molecules known as nucleotides. The term can be used to refer to a monomeric unit that is present in a polymer, for example to identify a subunit present in a DNA or RNA strand. The term can also be used to refer to a molecule that is not necessarily present in a polymer, for example, a molecule that is capable of being incorporated into a polynucleotide in a template dependent manner by a polymerase. The term can refer to a nucleoside unit having, for example, 0, 1, 2, 3 or more phosphates on the 5′ carbon. For example, tetraphosphate nucleotides, pentaphosphate nucleotides, and hexaphosphate nucleotides can be particularly useful, as can nucleotides with more than 6 phosphates, such as 7, 8, 9, 10, or more phosphates, on the 5′ carbon. Example natural nucleotides include, without limitation, ATP, UTP, CTP, and GTP (collectively NTP), and ADP, UDP, CDP, and GDP (collectively NDP), or AMP, UMP, CMP, or GMP (collectively NMP), or dATP, dTTP, dCTP, and dGTP (collectively dNTP), and dADP, dTDP, dCDP, and dGDP (collectively dNDP), and dAMP, dTMP, dCMP, and dGMP (dNMP). Example nucleotides may include, without exception, any NMP, dNMP, NDP, dNDP, NTP, dNTP, and other NXP and dNXP where X represents a number from 2 to 10 (collectively NPP).

Non-natural nucleotides also referred to herein as nucleotide analogs, include those that are not present in a natural biological system or not substantially incorporated into polynucleotides by a polymerase in its natural milieu, for example, in a non-recombinant cell that expresses the polymerase. Particularly useful non-natural nucleotides include those that are incorporated into a polynucleotide strand by a polymerase at a rate that is substantially faster or slower than the rate at which another nucleotide, such as a natural nucleotide that base-pairs with the same Watson-Crick complementary base, is incorporated into the strand by the polymerase. For example, a non-natural nucleotide may be incorporated at a rate that is at least 2 fold different—e.g., at least 5 fold different, 10 fold different, 25 fold different, 50 fold different, 100 fold different, 1000 fold different, 10000 fold different or more when compared to the incorporation rate of a natural nucleotide. A non-natural nucleotide can be capable of being further extended after being incorporated into a polynucleotide. Examples include, nucleotide analogs having a 3′ hydroxyl or nucleotide analogs having a reversible terminator moiety at the 3′ position that can be removed to allow further extension of a polynucleotide that has incorporated the nucleotide analog. Examples of reversible terminator moieties that can be used are described, for example, in U.S. Pat. Nos. 7,427,673; 7,414,116; and 7,057,026 and PCT publications WO 91/06678 and WO 07/123744. It will be understood that in some examples a nucleotide analog having a 3′ terminator moiety or lacking a 3′ hydroxyl (such as a dideoxynucleotide analog) can be used under conditions where the polynucleotide that has incorporated the nucleotide analog is not further extended. In some examples, nucleotide(s) may not include a reversible terminator moiety, or the nucleotides(s) will not include a non-reversible terminator moiety or the nucleotide(s) will not include any terminator moiety at all. Nucleotide analogs with modifications at the 5′ position are also useful.

A “primer” is defined as a single stranded nucleic acid sequence (e.g., single strand DNA or single strand RNA) that serves as a starting point for DNA or RNA synthesis or, in the case of an immobilized primer, a template for extension of a test primer. The 5′ terminus of a primer for affixation to a surface may be modified to allow a coupling reaction with a functionalized layer or functionalized polymer layer on a surface. The primer length can be any number of bases long and can include a variety of non-natural nucleotides. In an example, the primer is a short strand, ranging from 20 to 40 bases, or 10 to 20 bases.

In some examples, immobilized primers may be attached directly to a surface or substrate or functionalized surface of a substrate or substrate. In other example, a surface or substrate may be further modified by addition of polymers attached to the surface or substrate and immobilized primers attached to the surface or substrate via attachment to such polymers. Such polymers may be random, block, linear, and/or branched copolymers comprising two or more recurring monomer units in any order or configuration, and may be linear, cross-linked, or branched, or a combination thereof. In an example, a polymer used may include examples such as poly(N-(5-azidoacetamidylpentyl)acrylamide-co-acrylamide), also known as PAZAM. In an example, the polymer may be a heteropolymer and the heteropolymer may include an acrylamide monomer, such as

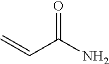

or a substituted analog thereof (“substituted” referring to the replacement of one or more hydrogen atoms in a specified group with a another atom or group). In some examples, the acrylamide monomer may include an azido acetamido pentyl acrylamide monomer:

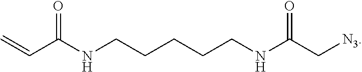 [SMILES: C=CC(=O)NCCCCCNC(=O)CN=[N+]=[N-]]

In some examples, the acrylamide monomer may include an N,N-dimethylacrylamide

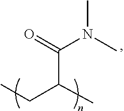

wherein n corresponds to y in examples including x-y copolymers, and wherein n corresponds to z in examples including x-y-z copolymers.

In an example, the polymer is a heteropolymer and may further include an azido-containing acrylamide monomer. In some aspects, the heteropolymer includes:

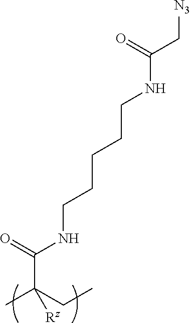

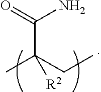

and optionally

In some aspects, the heteropolymer may include the structure:

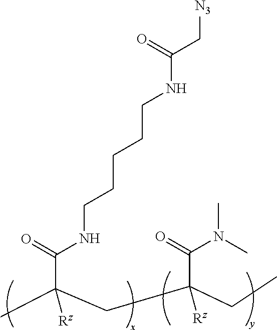

where each R^{z} is independently H or C₁₋₄ alkyl, which structure may be referred to herein as an “x-y copolymer.” In some examples, a ratio of x:y may be from approximately 15:85 to approximately 1:99, e.g., from approximately 10:90 to approximately 1:99, e.g., from approximately 10:90 to approximately 5:99, or may be approximately 5:95. In other aspects, the heteropolymer may include the structure:

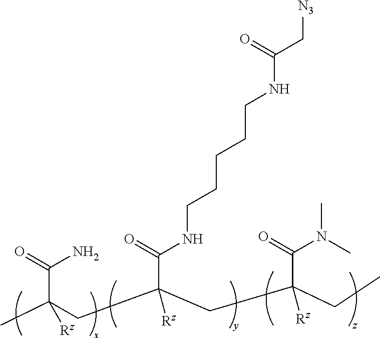

where each R^{z} is independently H or C₁₋₄ alkyl, which structure may be referred to herein as an “x-y-z copolymer.” In some examples, a ratio of (x:y):z may be from approximately 85:15 to approximately 95:5, or may be approximately 90:10 (wherein a ratio of x:(y:z) may be from approximately 1:(99) to approximately 10:(90), or may be approximately 5:(95)), respectively. In some examples, a ratio of x:y:z may be from approximately 0:15:85 to approximately 0:5:95. In an example, a ratio of x:y is 5:95. In another example, a ratio of x:y:z is 5:85:10. In these examples, approximately means relative amounts of one may differ from amounts stated in the listed rations by up to 5%.

A “heteropolymer” is a large molecule of at least two different repeating subunits (monomers). An “acrylamide monomer” is a monomer with the structure

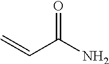

or a substituted analog thereof (e.g., methacrylamide or N,N-Dimethylacrylamide). An example of a monomer including an acrylamide group and the azido group is azido acetamido pentyl acrylamide shown above. “Alkyl” refers to a straight or branched hydrocarbon chain that is fully saturated (i.e., contains no double or triple bonds). Example alkyl groups include methyl, ethyl, dimethylacrylamide, propyl, isopropyl, butyl, isobutyl, and tertiary butyl. As an example, the designation “C₁₋₄ alkyl” indicates that there are one to four carbon atoms in the alkyl chain, i.e., the alkyl chain is selected from the group consisting of methyl, ethyl, dimethyl, propyl, iso-propyl, n-butyl, isobutyl, sec-butyl, and t-butyl.

In some examples, it may be desirable to both measure an amount of hybridizable immobilized primer or primers on a substrate and also polymerase accessible immobilized primer in a method as disclosed herein. An example of such a method is depicted in brief in FIG. 8A. Here, an immobilized primer is shown with its 3-prime end extending distally from the surface to which it is covalently attached. A complementary, test primer hybridized thereto is also shown. In this example, one fluorophore, indicated with a dark star, is shown at the test primer's 5-prome end and another fluorophore, as a lighter star, is shown on the test primer's 3-prome end. In this example, the test primer possessed a 5-prime fluorophore when added to the surface during incubation and hybridization. At that point, the method resembles conventional methodology for measuring hybridization-accessible immobilized primers. By measure fluorescence emitted from a surface after incubation with such a test primer having a fluorophore previously attached to its 5-prime end one can determine hybridization-accessible immobilization primers.

However, furthermore in this example, the test primer-immobilized primer pair is further incubated with a polymerase and nucleotides with an attached fluorophore, wherein the fluorescently tagged nucleotide is attachable to the 3-prime end of a test primer by a polymerase using the immobilized primer as a template, much as described above. The result is the test primer-immobilized primed paid illustrated in FIG. 8A. There, the test primer is shown with the 5-prime fluorophore to which it was initially attached, as well as the 3-prime fluorophore attached thereto by a polymerase. By measuring the amount of the first fluorophore an amount of hybridization accessible immobilized primer present on the surface may be determined. But measuring how much of the fluorophore attached to the 3-prime-most nucleotide is present (in accordance with examples as disclosed above), an amount of polymerase-accessible immobilized primer may be determined. By combining both measurements in one assay in accordance with this example, more information can be obtained more efficiently. In this example, the 5-prime fluorophore and the 3-prime fluorophore added by polymerase are detectably different from each other.

Examples disclosed above include examples of a method wherein an immobilized primer is not covalently modified. Such examples may be of particular use where, for example, a substrate with immobilized primers is intended for use after an assay in accordance with a method disclosed herein has been completed and no covalent modifications to the immobilized primers is desired. In other example, however, a method as disclosed herein may include covalent modification of immobilized primers. Three such examples are illustrated in FIGS. 8B and 8C. In FIG. 8B, a test primer is shown with a 5-prime overhang, which can serve as a template for extension not only of the test primer at its 3-prime end in accordance with the above disclosure, but also now of the 3-prime end of the immobilized primer, distal to the surface. In such an example, a test primer and immobilized primer may be designed such that detectably different nucleotides may be incorporated into the 3-prime end of each.

In another example, such as shown in FIG. 8C, a 5-prime end of test primers may include a fluorescently labeled nucleotide. Such fluorescently labeled nucleotide may overhang the immobilized primer and thereby serve as a template for extension thereof. In this example, the fluorophore attached to the 5-prime end of the test primer and the fluorophore attached to the nucleotide added to the 3-prime end of the immobilized primer using the 5-prime overhang of the test primer as a template are detectably different from each other. In some examples, proximity of these two fluorophores to each other may influence fluorescence output. For example, according to fluorescence quenching, fluorophores may be selected such that emissions from either or both of the pair is blunted by quenching when the fluorophores are in such proximity to each other as may occur when a labeled nucleotide is added to the 3-prime end of the immobilized primer using the test primer as a template.

Under such circumstances, for example where the 3-prime added fluorescent nucleotide quenches fluorescent emission from the 5-prime attached fluorophore of the test primer, reduction of fluorescence emitted from the 5-prime test primer fluorophore may be taken as an indication that a nucleotide has been added, such as by a polymerase, to the 3-prime end of the immobilized primer. Alternatively, measurement of an increase in fluorescence emission from the immobilized primer upon dehybridization of the test primer may indicate that a polymerase had catalyzed the addition of a fluorescent nucleotide to the 3-prime end of the immobilized primer. Alternatively, the fluorophores in this example may be selected so as to undergo fluorescent resonance energy transfer such that one serves as a donor to the other to stimulate florescence by the other when they are in such proximity to each other as may occur when a polymerase catalyzes the addition of a fluorescently tagged nucleotide to the 3-prime end of the immobilized primer. In that case, detection of the emission characteristic of the occurrence of fluorescent resonance energy transfer between the fluorophores may indicate that a nucleotide had been added by a polymerase to the 3-prime end of the immobilized primer. Alternatively, loss of such emission upon dehybridization of the test primer may indicate that a nucleotide had been added by a polymerase to the immobilized primer. Examples of detecting an amount of fluorescent test primers that that include detecting the combined fluorescence emitted by a pair of fluorescent nucleotides include such examples where detection includes detecting quenching or fluorescent resonance energy transfer.

Other variations, combinations, or modifications of the foregoing examples are also within the scope of the present disclosure. The foregoing examples are intended to illustrate examples of the present disclosure, but are by no means intended to limit the scope thereof. Although examples have been depicted and described in detail herein, it will be apparent to those skilled in the relevant art that various modifications, additions, substitutions, and the like can be made without departing from the spirit of the present disclosure and these are therefore considered to be within the scope of the present disclosure.

1. A method, comprising
hybridizing test primers to immobilized primers; wherein the immobilized primers comprise a predetermined sequence of nucleotides and are attached to a substrate through their 5-prime ends, individual test primers are complementary to a portion of each of at least some of the immobilized primers, and no more than one test primer molecule hybridizes to an immobilized primer molecule,
extending, using one nucleotide, at least some of the test primers with a polymerase according to templates; wherein said templates comprise immobilized primers hybridized to said at least some of the test primers, and nucleotides incorporated into said at least some of the test primers by the extending comprise one of a plurality of fluorescent tags, and
detecting an amount of fluorescent test primers.

2. The method of claim 1, wherein nucleotide sequences of a first plurality of the immobilized primers differ from nucleotide sequences of a second plurality of the immobilized primers, a first plurality of the test primers is complementary to a portion of the first plurality of immobilized primers, and a second plurality of the test primers is complementary to a portion of the second plurality of immobilized primers.

3. (canceled)

4. The method of claim 2, wherein first nucleotides incorporated into the first plurality of the test primers comprise a first of the plurality of fluorescent tags, second nucleotides incorporated into the second plurality of the test primers comprise a second of the plurality of fluorescent tags, and fluorescence emitted by the first of the plurality of fluorescent tags differs from fluorescence emitted by the second of the plurality of fluorescent tags.

5. (canceled)

6. (canceled)

7. (canceled)

8. (canceled)

9. The method of claim 1, wherein the substrate further comprises a polymer, at least some of the immobilized primers are attached to the polymer, and the polymer is a heteropolymer selected from 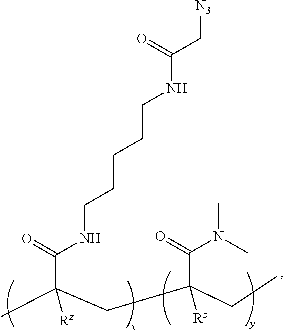
wherein x and y are integers representing a number of monomers and a ratio of x:y may be from approximately 5:85 to approximately 1:99, and 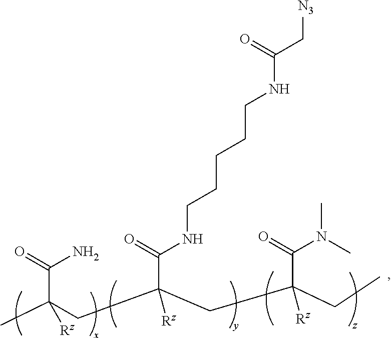
wherein x, y, and z are integers representing a number of monomers and a ratio of (x:y):z may be from approximately (85):15 to approximately (95):5, and wherein each R^{z} is independently H or C₁₋₄ alkyl.

10. The method of claim 9, wherein the heteropolymer comprises 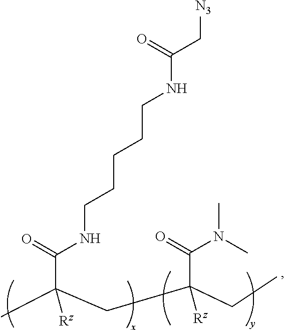
wherein the ratio of x:y is approximately 10:90.

11. The method of claim 9, wherein the heteropolymer comprises 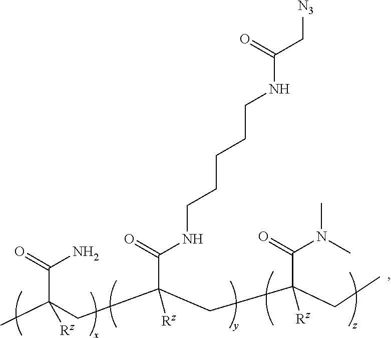
wherein the ratio of x:y:z is approximately 5:85:10.

12. (canceled)

13. The method of claim 1, wherein said polymerase is attached to the substra

14. The method of claim 1, wherein said polymerase is not attached to the substrate.

15. (canceled)

16. (canceled)

17. (canceled)

18. The method of claim 1, wherein the detecting comprises measuring fluorescence emitted from test primers.

19. The method of claim 18, wherein test primers are hybridized to immobilized primers during the measuring.

20. The method of claim 18, further comprising dehybridizing the test primers from the immobilized primers before the measuring.

21. The method of claim 3, wherein the detecting comprises measuring fluorescence emitted from the test primers in response to a stimulus.

22. The method of claim 21, wherein test primers are hybridized to immobilized primers during the measuring.

23. The method of claim 21, further comprising dehybridizing the test primers from the immobilized primers before the measuring.

24. The method of claim 12, further comprising comparing an amount detected of the first of the plurality of fluorescent tags to an amount detected of the second of the plurality of fluorescent tags.

25. The method of claim 1, wherein a 5-prime end of at least some of the test primers does not overhang 3-prime ends of the immobilized primers.

26. The method of claim 1, wherein a 5-prime end of at least some of the test primers comprises a 5-prime fluorescent tag having a 5-prime fluorescent tag spectrum, the 5-prime fluorescent spectrum differs from the fluorescent spectrum of the fluorescent tag of the nucleotide incorporated into the test primer by the extending, and detecting comprises detecting an amount of 5-prime fluorescent tag and an amount of nucleotide fluorescent tag.

27. The method of claim 1, wherein at least some of the test primers comprise overhanging test primers complementary to overhung immobilized primers, wherein 5-prime ends of the overhanging test primers overhang 3-prime ends of the overhung immobilized primers when hybridized thereto before the extending, further comprising
extending, using one nucleotide, overhung immobilized primers, wherein nucleotides incorporated into overhung immobilized primers by the extending comprise an overhung fluorescent tag having an emission spectrum detectably different from an emission spectrum of the one of a plurality of fluorescent tags incorporated into the overhanging test primer by the extending,
detecting an amount of fluorescent immobilized primers, and
comparing an amount of fluorescent test primers to an amount of fluorescent immobilized primers.

28. The method of claim 1, wherein at least some of the test primers comprise overhanging test primers complementary to overhung immobilized primers, wherein 5-prime ends of the overhanging test primers overhang 3-prime ends of the overhung immobilized primers when hybridized thereto before the extending and comprise a test primer 5-prime fluorescent tag, further comprising
extending, using one nucleotide, overhung immobilized primers, wherein nucleotides incorporated into overhung immobilized primers by the extending comprise an overhung fluorescent tag, the test primer 5-prime fluorescent tag and the overhung fluorescent tag comprise a fluorescent tag pair when the overhanging test primers are hybridized to overhung immobilized primers after the extending of the overhung immobilized primers, and a combined fluorescence emitted by the fluorescent tag pair differs from a fluorescence emitted from the test primer 5-prime fluorescent tag and from a fluorescence emitted from the overhung fluorescent tag.

29. The method of claim 28, wherein detecting an amount of fluorescent test primers comprises detecting the combined fluorescence emitted by the fluorescent tag pair.


SEQUENCE LISTING (2 sequences)

SEQ ID NO: 1 - DNA, 24 bp, Artificial Sequence
  Description of Artificial Sequence Synthetic primer
caagcagaagacggcatacgagat

SEQ ID NO: 2 - DNA, 29 bp, Artificial Sequence
  Description of Artificial Sequence Synthetic primer
aatgatacggcgaccaccgagatctacac